(19) United States
(12) Patent Application Publication
(10) Pub. No.: US 2009/0075354 A1
(43) Pub. Date: Mar. 19, 2009

(54) NANOFIBER STRUCTURES FOR SUPPORTING BIOLOGICAL MATERIALS

(75) Inventors: Darrell H. Reneker, Akron, OH (US); Daniel J. Smith, Stow, OH (US)

Correspondence Address: ROETZEL AND ANDRESS, 222 SOUTH MAIN STREET, AKRON, OH, 44308, US

(73) Assignee: THE UNIVERSITY OF AKRON, Akron, OH (US)

(21) Appl. No.: 11/916,294

(22) PCT Filed: Jun. 7, 2006
(86) PCT No.: PCT/US2006/021785

§ 371 Date: Oct. 16, 2008

Related U.S. Application Data
(60) Provisional application No. 60/688,025, filed on Jun. 7, 2005.

(51) Int. Cl. C12N 11/04; C07K 14/00; D02G 3/44; C07H 21/00

(52) U.S. Cl. 435/182; 530/350; 536/22.1; 428/401

(57) ABSTRACT

The present invention relates generally to nanofiber structures designed to support, entrap, entangle, preserve, and/or retain one or more biological materials. More specifically, the present invention relates to nanofiber matrix structures made from at least two different types of nanofibers that are designed to support, entrap, entangle, preserve, and/or retain one or more biological materials.

### FIELD OF THE INVENTION

The present invention relates generally to nanofiber structures designed to support, entrap, entangle, preserve, and/or retain one or more biological materials. More specifically, the present invention relates to nanofiber matrix structures made from at least two different types of nanofibers that are designed to support, entrap, entangle, preserve, and/or retain one or more biological materials.

### BACKGROUND OF THE INVENTION

Biological materials may be preserved for long term storage by a number of techniques including storage at low temperatures and freeze-drying. Storage at low temperature, while effective, is limited to applications where constant refrigeration is available. The need for constant refrigeration limits the usefulness of this technique. Preservation of biological samples by freeze-drying, however, is not so limited.

The technique of freeze-drying, also known as lyophilization, involves the freezing of a sample, forming water crystals, followed by the direct sublimation of the water crystals, usually under vacuum. That is, the water is directly converted from a solid state to a gaseous state without passing through a liquid state. Freeze-drying, therefore, typically dehydrates a sample without denaturing or otherwise altering its three-dimensional structure by heating. Once freeze-dried, samples are often stable at room temperature for an extended period of time provided that the samples are stored in a water-vapor impermeable container, such as, for example, a glass ampule. Therefore, freeze-drying provides a method of long term storage of biological materials at room temperature.

Freeze-drying, however, has disadvantages associated with it. Freeze-drying requires both time and expensive equipment. Freeze-drying can also cause irreversible changes to occur in some proteins or other samples by mechanisms other than those associated with heating. Among these changes are denaturation caused by a change in pH or by the concentration of other substances near the molecules of the biological material. Therefore, there is a need for a method of preservation of biological materials that provides an alternative to freeze-drying. Such a need is acutely felt with regard to the delivery of biological materials to remote areas requiring long transport times with little or no refrigeration available. The delivery of vaccines or other medical products to remote areas is one specific example of such a need. Ideally, such a method would provide an economical method for long term preservation of such samples at room temperature.

The technique of electrostatic spinning, also known within the fiber forming industry as electrospinning, of liquids and/or solutions capable of forming fibers, is well known and has been described in a number of patents, such as, for example, U.S. Pat. Nos. 4,043,331 and 5,522,879 (incorporated herein by reference in their entireties for their teachings of electrospinning techniques). The process of electrostatic spinning generally involves the introduction of a liquid into an electric field, so that the liquid is caused to produce fibers. These fibers are generally drawn to a conductor at an attractive electrical potential for collection. During the conversion of the liquid into fibers, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening). The process of electrostatic spinning has typically been directed toward the use of the fibers to create a mat or other non-woven material, as disclosed, for example, in U.S. Pat. No. 4,043,331. In other cases, electrospinning is used to form medical devices such as wound dressings, vascular prostheses, or neural prostheses as disclosed, for example, in U.S. Pat. No. 5,522,879.

### SUMMARY OF THE INVENTION

The present invention relates generally to nanofiber structures designed to support, entrap, entangle, preserve, and/or retain one or more biological materials. More specifically, the present invention relates to nanofiber matrix structures made from at least two different types of nanofibers that are designed to support, entrap, entangle, preserve, and/or retain one or more biological materials.

In one embodiment, the present invention relates to a method of preserving at least one biological material comprising the steps of: (A) providing at least one water-soluble fiber-forming material; (B) mixing at least one biological material, and optionally, one or more additives, with the at least one water-soluble fiber-forming material to form a mixture; (C) forming at least one water-soluble fiber layer/structure from the mixture, wherein the one or more fibers of the water-soluble layer/structure have a diameter between about 0.1 nanometers and about 25,000 nanometers; (D) providing at least one water-insoluble fiber-forming material, the at least one water-insoluble fiber-forming material optionally including one or more additives; and (E) forming at least one water-insoluble fiber layer/structure that is in contact with at least one surface of the at least one water-soluble fiber layer/structure, wherein the one or more fibers of the water-insoluble layer/structure have a diameter between about 0.1 nanometers and about 25,000 nanometers.

In another embodiment, the present invention relates to a biological material preserved by/via the above method.

In still another embodiment, the present invention relates to a structure supporting and preserving at least one biological material, the structure comprising: a first fiber layer, the first fiber layer having an upper surface and a lower surface, wherein the first fiber layer is formed from at least one water-soluble fiber-forming material and wherein the first fiber layer contains, supports, entraps, entangles, preserves, and/or retains the at least one biological material; and a second fiber layer, the second fiber layer having an upper surface and a lower surface, wherein the lower surface of the second fiber layer is in contact with the upper surface of the first fiber layer and wherein the second fiber layer is formed from at least one water-insoluble fiber-forming material.

In still another embodiment, the present invention relates to a structure supporting at least one biological material, the structure comprising: a first fiber layer, the first fiber layer having an upper surface and a lower surface, wherein the first fiber layer is formed from at least one water-soluble fiber-forming material and wherein the first fiber layer contains, supports, entraps, entangles, preserves, and/or retains the at least one biological material; and a second fiber layer, the second fiber layer having an upper surface and a lower surface, wherein the lower surface of the second fiber layer is in contact with the upper surface of the first fiber layer and wherein the second fiber layer is formed from at least one water-insoluble fiber-forming material, and wherein the one or more fibers of the first fiber layers have a diameter between about 0.1 nanometers and about 25,000 nanometers, and wherein the one or more fibers of the second fiber layers have a diameter between about 0.1 nanometers and about 25,000 nanometers.

### BRIEF DESCRIPTION OF THE DRAWINGS

FIG. 1 is an illustration of one embodiment of a polymer nanofiber structure according to the present invention;

FIG. 2 is an illustration of another embodiment of a polymer nanofiber structure according to the present invention; and

FIG. 3 is an illustration of yet another embodiment of a polymer nanofiber structure according to the present invention.

### DETAILED DESCRIPTION OF THE INVENTION

As mentioned above, the present invention relates generally to nanofiber structures designed to support, entrap, entangle, preserve, and/or retain one or more biological materials. More specifically, the present invention relates to nanofiber matrix structures made from at least two different types of nanofibers that are designed to support, entrap, entangle, preserve, and/or retain one or more biological materials.

In one embodiment the present invention relates to a nanofiber structure formed from a combination of nanofibers formed from at least one water-soluble polymer and nanofibers formed from at least one water-insoluble polymer. The water-insoluble polymer can possess a wide variety of chemical and/or physical properties. For example, the water-insoluble polymer of the present invention could be soluble in other types of solvents (e.g., alcohols, etc.), be bioactive, biodegradable, elastometric, electrically conductive, etc.

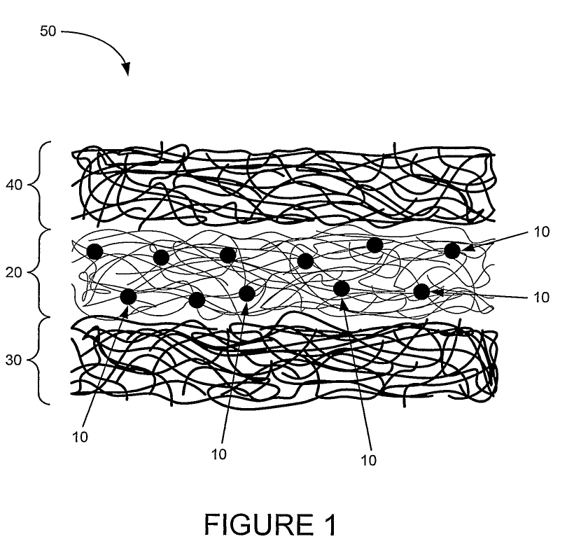

In this embodiment, as is shown in FIG. 1, the biological material 10 is supported, entrapped, entangled, preserved, and/or retained in a nanofiber structure 20 formed from the water-soluble polymer. The water-soluble polymer/biological material combination is then supported, entrapped, entangled, preserved, encased, and/or retained by one or more nanofiber structures 30, 40 formed from at least one water-insoluble polymer. Taken together, the three layers form an overall nanofiber structure 50 that supports, entraps, entangles, preserves, and/or retains one or more biological materials. With regard to the thickness and/or darkness of the lines in FIG. 1 used to represent the fibers that make up each of layers 20, 30 and 40, the thickness of the lines is only used to differentiate between layers and do not have any meaning with regard to the diameters of the fiber in each of layers 20, 30 and 40.

It should be noted that although the fibers in each portion 20, 30 and 40 of structure 50 are shown at different thicknesses and lengths, the present invention is not limited thereto. In fact, the present invention can include nanofiber structures of any length, so long as the fibers included in the present invention have diameters in the range of about 0.1 nanometers to about 25,000 nanometers.

In another embodiment, the nanofibers of the present invention are fibers having an average diameter in the range of about 1 nanometer to about 25,000 nanometers (25 microns), or about 1 nanometer to about 10,000 nanometers, or about 1 nanometer to about 5,000 nanometers, or about 3 nanometers to about 3,000 nanometers, or about 7 nanometers to about 1,000 nanometers, or even about 10 nanometers to about 500 nanometers. In another embodiment, the nanofibers of the present invention are fibers having an average diameter of less than 25,000 nanometers, or less than 10,000 nanometers, or even less than 5,000 nanometers. In still another embodiment, the nanofibers of the present invention are fibers having an average diameter of less than 3,000 nanometers, or less than about 1,000 nanometers, or even less than about 500 nanometers. Additionally, it should be noted that here, as well as elsewhere in the text, ranges may be combined.

Furthermore, the diameters of the fibers in each portion 20, 30 and 40 of structure 50 can be independently chosen from the range of fiber diameters mentioned above.

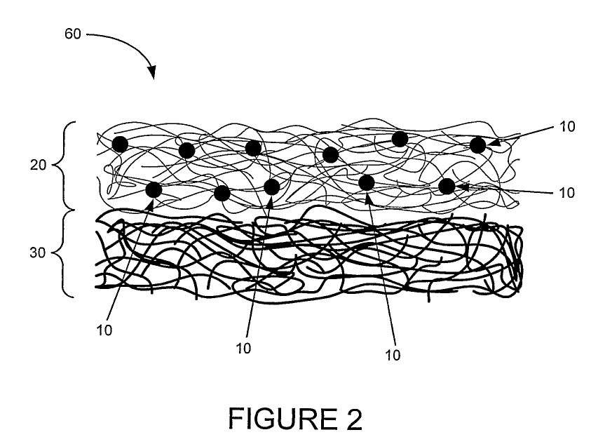

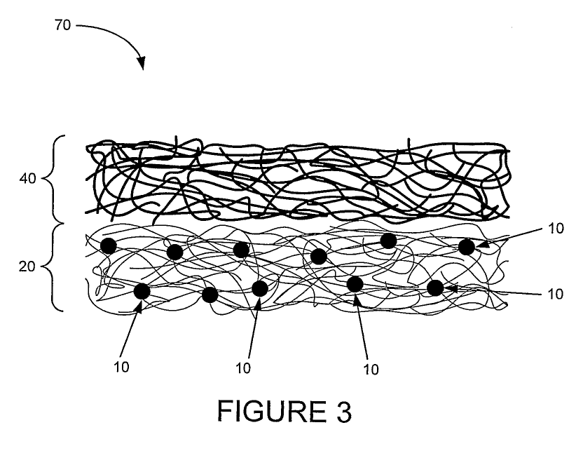

In another embodiment, structure 50 can contain two layers so long as one of the two layers is formed from a water-soluble polymer and includes therein at least one biological material. For example, layer 40 or layer 30 could be eliminated in this embodiment. In this regard, FIGS. 2 and 3 illustrate embodiments where layers 40 and 30, respectively, have been eliminated from the structure of FIG. 1. As can be seen in FIGS. 2 and 3, structures 60 and 70, respectively, are two layer structures.

The mixture of biological material and the water-soluble fiber-forming material for layer 20 can be formed into fibers by any method which does not negatively affect the activity of the biological material such as by heating, for example. Such methods include electrospinning and the “Nanofibers by Gas Jet” or NGJ technique disclosed in U.S. Pat. No. 6,382,526 (incorporated herein by reference in its entirety).

With regard to fiber layers 30 and 40, these layers can also be formed by any suitable fiber forming method which permits the formation of fibers having diameters within the range stated above. Such methods include, for example, electrospinning and NGJ.

Electrospinning generally involves the introduction of a polymer or other fiber-forming liquid into an electric field, so that the liquid is caused to produce fibers. These fibers are drawn to an electrode at a lower electrical potential for collection. During the drawing of the liquid, the fibers rapidly harden and/or dry. The hardening/drying of the fibers may be caused by cooling of the liquid, i.e., where the liquid is normally a solid at room temperature; by evaporation of a solvent, e.g., by dehydration (physically induced hardening); by a curing mechanism (chemically induced hardening); or by a combination of these methods. Electrostatically spun fibers can be produced having very thin diameters.

It will be appreciated that, because of the very small diameter of the fibers, the fibers have a high surface area per unit of mass. This high surface area to mass ratio permits fiber-forming material solutions to be transformed from solvated fiber-forming materials to solid nanofibers in fractions of a second. When biological materials are dissolved or suspended in a water-soluble fiber-forming material solution which is then formed into water-soluble fibers, the samples experience a rapid loss of excess solvent. This invention thereby also provides a fiber containing a substantially homogeneous mixture of at least one fiber-forming material and at least one preserved biological material. While not wishing to condition patentability on any particular theory of operation, it is believed that in the same time interval in which destabilizing changes such as changes in pH or concentration occur, these samples become embedded in a fibrous polymer matrix which immobilizes and protects the sample. Alternatively or in addition to, at least a portion of the biological sample embedded in the matrix may reversibly denatured to some degree and re-natured in an active conformation upon re-hydration. It is believed, therefore, that the fiber of the present invention contains biological material embedded in a dry protective matrix. It should be understood however, that while the fiber is described herein as being “dry”, the biological material may retain a certain amount of water provided that the water present does not interfere with the solidification of the fiber. That is, formation of a dry fiber should be understood as not precluding the association of water of hydration with the biological sample to form a hydrate solid.

The at least one water-soluble fiber-forming material used in this invention can be selected from any water-soluble fiber-forming material which can be dissolved and is otherwise compatible with the biological material to be preserved. Water-soluble fiber-forming materials which may be used in the practice of the method of the present invention include, but are not limited to, the following water-soluble polymers: poly (vinyl pyrrolidone) (PVP), polyethyl oxazoline (PEOZ), polyethylenimine (PEI), polyethylene oxide (PEO) and mixtures of two or more thereof.

The at least one water-insoluble fiber-forming material used in this invention can be selected from any water-insoluble fiber-forming material that can be formed, via any suitable method, into fibers. Water-insoluble fiber-forming materials which may be used in the practice of the method of the present invention include, but are not limited to, the following water-insoluble polymers: polyolefin polymers (e.g., Tyvek®, polyethylene, polystyrene, etc.), cellulose polymers (e.g., carboxymethyl cellulose (CMC)), polyvinyl polypyrrolidone (PVPP), water-insoluble starch-based polymers (e.g., glucose polymers in which glucopyranose units are bonded by alpha-linkages), Nafion® (a sulfonated tetrafluorethylene copolymer), and mixtures of two or more thereof. In still another embodiment, the water-insoluble polymer is biocompatible and/or biodegradable.

In one embodiment, the structures of the present invention are formed via an electrospinning and/or NGJ process that utilize a solvent that dissolves and/or solubilizes the at least one fiber-forming material but does not dissolve and/or solubilize the one or more biological material. As an example, one could take DNA or an enzyme, suspend the dry material in ethanol and mix it with linear polyethylenimine. In this example, the polymer dissolves, but the biological does not. Thus, the polymer in this case can be spun out, with the one or more biological materials becoming entrapped or encased within the fiber. It should be noted that the present invention is not limited to just the above example.

It is envisioned that the present invention will typically be used to preserve a biological material for later use. Upon completion of the preservation period, the biological material is recovered from the water-soluble fiber by the application, introduction and/or presence of water or water vapor. Alternatively, another solvent can be used, provided that the solvent is compatible with the preserved biological material. Other methods for recovering the biological material from the fiber are also envisioned. These include biodegradation, hydrolysis, thermal melting or other de-polymerization of the fiber-forming material. Upon recovery, the biological material must possess at least a portion of its original biological activity. In one embodiment, the biological material preserved in the nanofiber structure 50 of the present invention should retain at least about 25, about 30, about 40, about 50, about 60, about 70, about 80, about 90 or even at least about 95 percent of its activity when stored at room temperature (approximately 20 to 25° C.) for at least about 12 hours, about 24 hours, about 48 hours, about 1 week, about 15 days, about 1 month, or even at least about 6 months or about 12 months.

Biological materials which may be a component of fiber structure 10 of the present invention generally include, by way of example and not of limitation, proteinaceous compounds, carbohydrates, nucleic acids and mixtures thereof.

Non-limiting examples of proteinaceous compounds which may be utilized in the fiber of the present invention include peptides, polypeptides, proteins, enzymes, coenzymes, holoenzymes, enzyme subunits, and prions. Enzymes which may be used include peroxidase, trypsin, and thrombin, although other enzymes may also be used. The fiber of the present invention maybe spun to form mats of fiber containing at least one fiber-forming material and at least one biological material. When thrombin or any other medically useful protein is utilized, the fiber of the present invention may be a component of a medical dressing or other medical device. Other therapeutic compounds, including therapeutic peptides or polypeptides, may be present in the fiber. Examples include viral fusion inhibitors, hormone antagonists, and other compounds which exert a therapeutic effect by binding with a receptor molecule in vivo. Likewise, other viral proteins may also be used such as viral lytic proteins or other bacteriophage “killer” proteins. Other therapeutic proteins that have an adverse effect on pathogens are also envisioned as being preserved according to the present invention.

A non-limiting example of a carbohydrate that may be utilized in the present invention includes dextran. One or more carbohydrates such as glucose, fructose, or lactose, for example, may also be present to act as a stabilizer of another biological material such as an enzyme or other protein. Other additives, such as, for example, polyethylene glycol, may also be present.

Non-limiting examples of nucleic acids include ribonucleic acids and deoxyribonucleic acids. This includes ribonucleic acids such as anti-sense ribonucleic acid sequences and ribozymes, and deoxyribonucleic acids such as oligonucleotides, gene fragments, natural and artificial chromosomes, plasmids, cosmids, and other vectors. When incorporated into a dressing or other medical device, the vectors may encode for proteins such as the viral “killer” proteins mentioned above as an anti-infective agent. This includes vectors that encode lytic proteins that cause the target cells to rupture. Other proteins that interfere with target cell metabolism may also be encoded for by the vector.

It is envisioned that the at least one biological material may be a mixed sample containing more than one type of biological material. Additionally, the at least one biological material may be labeled with a marker such as, for example, a radioactive marker, a fluorescent marker, or a gold or other high atomic number particle which is visible by electronmicroscopy.

As mentioned above, the preserved biological material of the present invention may be a component of a medical dressing or other medical device. It is also envisioned that other therapeutic agents may be preserved according to this method, either for medical devices or as other structures. This includes bacteriophages, which are viruses that infect bacteria. Suitable bacteriophages, or simply phages, include those that infect bacteria from the following genera: Staphylococcus, Streptococcus, Escherichia, Salmonella, Clostridium, Pseudomonas, Proteus, Listeria, Vibrio, and Bacillus. Specific strains that may be targeted by phage include Staphylococcus aureus, Streptococcus pyogenes, Escherichia coli, Clostridium perfringens, Clostridium septicum, Pseudomonas aeruginosa, Proteus vulgaris, Vibrio vulniticus, Listeria monocytogenes, and Bacillus anthraxis. A wound dressing incorporating a bacteriophage would be particularly useful for the treatment of diabetic ulcers or other infections where a lack of blood flow makes effective treatment with systemic antibiotics difficult. However, treatment of infections in the absence of decreased blood flow may also be effectively treated with bacteriophage preserved according to the method of the present invention. This includes infections caused by virulent bacteria such as Group A Streptococci. Bacteriophage against microbes that cause food poisoning may also be preserved according to this method and incorporated into food packaging.

According to the method of this invention, any type of whole cells can be preserved. This includes bacterial cells (especially those that are non-virulent), blood cells, platelets, genetically engineered cells of any type, skin cells, stem cells, etc. Preserved bacterial cells may also be incorporated into a medical dressing to act as a competitor of a virulent bacteria strain. For example, U.S. Pat. No. 6,264,967 describes the use of microorganisms of the genus Brachybacterium to eliminate Staphylococcus aureus. The present invention may be used to preserve bacteria such as Bachybacterium to treat Staphylococcus aureus infections. The present invention may also be used to preserve microorganisms for other purposes.

For example, the at least one biological material may be a material that is capable of acting as an antigen by eliciting an immune response by an individual when exposed to the biological material. When this is the case, the biological material preserved by the present invention may also be a component of a vaccine. In such an embodiment, a medically acceptable fiber-forming material may be used to preserve the antigen for later re-hydration and use as a vaccine. In general, re-hydration of the fiber of the present invention may be accomplished by mixing the fiber with a solvent for the fiber-forming material. When the fiber is used to preserve an antigen for use in a vaccine, the solvent will optimally be a medically acceptable compound. Depending on the antigen and re-hydration solution used, the resulting vaccine may be an injectible or an ingestible vaccine. Other medically acceptable administration techniques may also be used with the resulting vaccine. As mentioned above, it is envisioned that a bacterial strain may be preserved according to the method of this invention. A preserved bacterial strain may also be included in a vaccine. In such a case, the bacterial vaccine may be either a live vaccine or a dead vaccine. In the case of a dead vaccine, cell viability is not a concern provided that the antigenicity of the biological material is maintained.

The present invention may also be used to produce a component of a test kit in which the preserved biological material may be subsequently used in performing a function of the kit. Non-limiting examples of such a kit include test kits which may be used to determine the presence of a specific chemical or biological compound in a test material. Such a kit may be used, for example, to test for the presence of a specific metabolite or other compound in a blood, serum, urine or other fluid sample from an individual for clinical or forensic purposes. Other sources of test material might also be used with such a kit. Such a kit may also be used to determine the presence of chemical compounds in environmental samples, for example. More than one biological material may be preserved together in such a kit. For example, an enzyme and coenzyme or cofactor for a particular reaction may be preserved either in separate fibers or in the same fiber.

The relative amounts of water-soluble fiber-forming material and biological material that may be present in fiber layer 20 of the present invention can vary. In one embodiment, the biological material comprises between about 1 and about 12 percent by weight to volume (w/v) of the mixture from which the water-soluble fiber is electrospun. In another example, the biological material comprises about 1 percent of the mixture or less. In still another example, the biological material may be about 0.25 percent, about 0.5 percent, about 0.75 percent, or about 1.0 percent of the mixture by weight to volume. It is envisioned that larger or smaller concentrations of biological material may also be utilized.

As mentioned above, fibers spun electrostatically can have a very small diameter. These diameters may be as small as 0.3 nanometers and are more typically between 3 nanometers and about 25,000 nanometers. In one embodiment, the fiber diameters are on the order of about 100 nanometers to about 25,000 nanometers, or even on the order of about 100 nanometers to about 1,000 nanometers. Such small diameters provide a high surface area to mass ratio of about 300 m²/g. Within the present invention, a fiber may be of any length. The term fiber should also be understood to include particles that are drop-shaped, flat, or that otherwise vary from a cylindrical shape.

In addition to the biological material 10 of layer 20, the present invention can also include various other compounds that are supported, entrapped, entangled, preserved, and/or retained in one or more of fiber layers 20, 30 and/or 40. Examples of such compounds include, but are not limited to, hormones, growth factors, nutrients, supplements, growth promoters, growth inhibitors, protein compounds, anti-scarring compounds, anti-bacterials, anti-fungals, anti-oxidants, UV protectants, etc.

As mentioned above, the process of electrostatic spinning generally involves the introduction of a liquid into an electric field, so that the liquid is caused to produce fibers. These fibers are generally drawn to an electrode for collection. During the drawing of the liquid, the fibers harden and/or dry. This hardening and/or drying may be caused by cooling of the liquid, i.e., where the liquid is normally a solid at room temperature; by evaporation of a solvent, e.g., by dehydration (physically induced hardening); or by a curing mechanism (chemically induced hardening). The hardened fibers are collected on a receiver such as, for example, a polystyrene or polyester net or a foil slide. As one skilled in the art will recognize, the fibers may be spun using a wide variety of conditions such as potential difference, flow rate, and gap distance. These parameters will vary with conditions such as humidity or other environmental conditions, the size of the biological material or other additive, the solution viscosity, the collection surface, and the polymer conductivity, among others.

The at least one fiber-forming material for each of the fiber layers 20, 30 and 40 of the present invention are, in one embodiment, in a liquid state when they are electrospun. This is particularly true of the at least one water-soluble polymer material used to form fiber layer 20 since at least one biological material 10 is included therewith.

Mixtures of the at least one water-soluble fiber-forming material and at least one biological material include mixtures where the biological material is soluble in the at least one water-soluble fiber-forming material in its liquid state and those mixtures in which the at least one biological material is insoluble in the at least one water-soluble fiber-forming material in its liquid state. When the biological material is insoluble in the at least water-soluble one fiber-forming material in its liquid state, the biological material may take the form of a suspension in the water-soluble fiber-forming material. Whether the biological material is soluble or insoluble in the water-soluble fiber-forming material, the biological material and the water-soluble fiber-forming material may be mixed by any method which forms a substantially homogeneous mixture, including, for example, mechanical shaking or stirring, although other methods may be used. As one skilled in the art will recognize, solubility of the biological material in the water-soluble fiber-forming material solution will depend on the characteristics of the material itself, as well as factors such as, for example, the requirements of the material for a specific pH range, osmolarity, or the presence of co-factors for the material.

Based upon the foregoing disclosure, it should now be apparent that electrospinning of biological materials with polymers will carry out the objects set forth hereinabove. It is, therefore, to be understood that any variations evident fall within the scope of the claimed invention and thus, the selection of specific component elements can be determined without departing from the spirit of the invention herein disclosed and described.

As used herein, the term “fiber” includes not only structures that are cylindrical, but also includes structures which vary from a cylindrical shape, such as for example, structures which are spherical, acicular, droplet shaped, or flattened or ribbon shaped. Other configurations are also possible. For example, the fiber of the present invention may appear “beaded” or otherwise vary from an entirely cylindrical configuration.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

What is claimed is:

1. A method of preserving at least one biological material comprising the steps of:
(A) providing at least one water-soluble fiber-forming material;
(B) mixing at least one biological material, and optionally, one or more additives, with the at least one water-soluble fiber-forming material to form a mixture;
(C) forming at least one water-soluble fiber layer/structure from the mixture, wherein the one or more fibers of the water-soluble layer/structure have a diameter between about 0.1 nanometers and about 25,000 nanometers;
(D) providing at least one water-insoluble fiber-forming material, the at least one water-insoluble fiber-forming material optionally including one or more additives; and
(E) forming at least one water-insoluble fiber layer/structure that is in contact with at least one surface of the at least one water-soluble fiber layer/structure, wherein the one or more fibers of the water-insoluble layer/structure have a diameter between about 0.1 nanometers and about 25,000 nanometers.

2. The method of claim 1, wherein the at least one water-soluble fiber-forming material is selected from one or more poly (vinyl pyrrolidone) polymers, polyethyl oxazoline polymers, polyethylenimine polymers, polyethylene oxide polymers, or mixtures of two or more thereof.

3. The method of claim 1, wherein the at least one water-insoluble fiber-forming material is selected from one or more polyolefin polymers, cellulose polymers, polyvinyl polypyrrolidone polymers, water-insoluble starch-based polymers, sulfonated tetrafluorethylene copolymers, or mixtures of two or more thereof.

4. The method of claim 1, wherein the step of forming at least one water-soluble fiber layer/structure from the mixture comprises electrospinning the combination of the at least one water-soluble fiber-forming material and the at least one at least one biological material.

5. The method of claim 1, wherein the at least one biological material is selected from one or more proteinaceous compounds, carbohydrates, nucleic acids and mixtures thereof.

6. The method of claim 1, wherein the preserved biological material retains at least 25 percent of its activity when stored at room temperature for at least 12 hours.

7. The method of claim 1, wherein the preserved biological material retains at least 25 percent of its activity when stored at room temperature for at least 1 week.

8. The method of claim 1, wherein, the at least one biological material is a protein.

9. The method of claim 1, wherein the at least one biological material is an enzyme.

10. The method of claim 1, wherein the at least one biological material is thrombin.

11. The method of claim 1, wherein the at least one biological material is a component of a medical dressing.

12. The method of claim 1, wherein the at least one biological material is selected from one or more viral fusion inhibitors, hormone antagonists, and compounds which exert an effect on an organism by binding with a receptor molecule in vivo.

13. A biological material preserved by the method according to claim 1.

14. A structure supporting and preserving at least one biological material, the structure comprising:
a first fiber layer, the first fiber layer having an upper surface and a lower surface, wherein the first fiber layer is formed from at least one water-soluble fiber-forming material and wherein the first fiber layer contains, supports, entraps, entangles, preserves, and/or retains the at least one biological material; and
a second fiber layer, the second fiber layer having an upper surface and a lower surface, wherein the lower surface of the second fiber layer is in contact with the upper surface of the first fiber layer and wherein the second fiber layer is formed from at least one water-insoluble fiber-forming material.

15. The structure of claim 14, wherein the one or more fibers of the first fiber layers have a diameter between about 0.1 nanometers and about 25,000 nanometers.

16. The structure of claim 14, wherein the one or more fibers of the second fiber layers have a diameter between about 0.1 nanometers and about 25,000 nanometers.

17. The structure of claim 14, wherein the at least one water-soluble fiber-forming material is selected from one or more poly (vinyl pyrrolidone) polymers, polyethyl oxazoline polymers, polyethylenimine polymers, polyethylene oxide polymers, or mixtures of two or more thereof.

18. The structure of claim 14, wherein the at least one water-insoluble fiber-forming material is selected from one or more polyolefin polymers, cellulose polymers, polyvinyl polypyrrolidone polymers, water-insoluble starch-based polymers, sulfonated tetrafluorethylene copolymers, or mixtures of two or more thereof.

19. The structure of claim 14, wherein the first and second fiber layers, and the one or more fibers contained therein, are independently formed via an electrospinning or NGJ process.

20. The structure of claim 14, wherein the at least one biological material is selected one or more proteinaceous compounds, carbohydrates, nucleic acids and mixtures thereof.

21. The structure of claim 14, wherein the preserved biological material retains at least 25 percent of its activity when stored at room temperature for at least 12 hours.

22. The structure of claim 14, wherein the preserved biological material retains at least 25 percent of its activity when stored at room temperature for at least 1 week.

23. The structure of claim 14, wherein, the at least one biological material is a protein.

24. The structure of claim 14, wherein the at least one biological material is an enzyme.

25. The structure of claim 14, wherein the at least one biological material is thrombin.

26. The structure of claim 14, wherein the at least one biological material is a component of a medical dressing.

27. The structure of claim 14, wherein the at least one biological material is selected from one or more viral fusion inhibitors, hormone antagonists, and compounds which exert an effect on an organism by binding with a receptor molecule in vivo.

28. A structure supporting at least one biological material, the structure comprising:
a first fiber layer, the first fiber layer having an upper surface and a lower surface, wherein the first fiber layer is formed from at least one water-soluble fiber-forming material and wherein the first fiber layer contains, supports, entraps, entangles, preserves, and/or retains the at least one biological material; and
a second fiber layer, the second fiber layer having an upper surface and a lower surface, wherein the lower surface of the second fiber layer is in contact with the upper surface of the first fiber layer and wherein the second fiber layer is formed from at least one water-insoluble fiber-forming material, and
wherein the one or more fibers of the first fiber layers have a diameter between about 0.1 nanometers and about 25,000 nanometers, and wherein the one or more fibers of the second fiber layers have a diameter between about 0.1 nanometers and about 25,000 nanometers.
